(19) United States
(12) Patent Application Publication
(10) Pub. No.: US 2025/0205371 A1
(43) Pub. Date: Jun. 26, 2025

(54) HYDROGEL MICROPARTICLES FOR EFFECTIVE RADIOISOTOPE LABELING AND A METHOD FOR THEIR PREPARATION

(71) Applicants: PUSAN NATIONAL UNIVERSITY INDUSTRY-UNIVERSITY COOPERATION FOUNDATION, Busan (KR); PUSAN NATIONAL UNIVERSITY HOSPITAL, Busan (KR)

(72) Inventors: Seung Yun YANG, Miryang-si (KR); Keunyoung KIM, Yangsan-si (KR); Sodam KIM, Miryang-si (KR); Injung CHOI, Miryang-si (KR)

(73) Assignees: PUSAN NATIONAL UNIVERSITY INDUSTRY-UNIVERSITY COOPERATION FOUNDATION, Busan (KR); PUSAN NATIONAL UNIVERSITY HOSPITAL, Busan (KR)

(21) Appl. No.: 19/001,418

(22) Filed: Dec. 25, 2024

(30) Foreign Application Priority Data
Dec. 26, 2023 (KR) 10-2023-0190985
Dec. 24, 2024 (KR) 10-2024-0195879

(51) Int. Cl. A61K 51/06; A61K 9/16

(52) U.S. Cl. CPC A61K 51/065; A61K 9/1694; A61K 2121/00

(57) ABSTRACT

The present invention relates to hydrogel microparticles for highly efficient radioisotope labeling and a method of preparing the same, and the hydrogel microparticles can be labeled with cationic radioisotopes that are highly useful in medicine, and homogeneous hydrogel microparticles can be manufactured through a single process, and hydrogel microparticles manufactured through freeze-drying have the advantage of being able to be stored and distributed for long periods of time.

### CROSS-REFERENCE TO RELATED APPLICATION

This application claims priority to Korean Patent Application Nos. 10-2023-0190985 filed on Dec. 26, 2023 and 10-2024-0195879 filed on Dec. 24, 2024, which are hereby incorporated by reference in their entirety.

### BACKGROUND OF THE DISCLOSURE

The present invention provides highly efficient hydrogel microparticles for radioisotope labeling and a method of preparing the same.

Cancer is one of the diseases with the highest mortality rate worldwide, and its causes are diverse, resulting in various forms of the disease. Because of this complexity and diversity, cancer treatment is a very difficult task, and as a result, cancer still maintains a high mortality rate. Existing cancer treatment methods include surgery, radiotherapy, and chemotherapy. These traditional treatments have limitations, especially in that they are difficult to target and attack cancer tissue. In addition, the effectiveness of the treatment is limited due to various side effects that occur during treatment and problems such as resistance to anticancer drugs.

For this reason, radiotherapy using radioisotopes is being introduced to overcome the limitations of nonspecific delivery of conventional cancer treatment methods. This approach enables targeted treatment by emitting radiation around cancer tissues and provides high precision and effective treatment results. However, the types of radioisotopes that can be accurately delivered to specific tissues are limited (e.g., 131-iodine), and there is a limitation that normal tissues can be affected by radioisotopes beyond the target area.

Recently, efforts have been made to overcome the limitations of radiotherapy using radioisotopes. To this end, the present inventors developed a technology to manufacture polymer-radioisotope conjugates through a process of binding radioisotopes to polymers, and formulate them into injectable microparticles, enabling localized radiation therapy. However, considering the half-life of radioisotope conjugates, this method can lead to loss of radioisotopes and decreased activity because it takes time to manufacture microparticles and set the injection dose.

### PRIOR ART DOCUMENT

### Non-Patent Document

Polymers 2020, 12, 1138 (published on May 16, 2020)

### SUMMARY OF THE DISCLOSURE

An object of the present invention is to provide hydrogel microparticles having high surface area for rapid and highly efficient on-site labeling of radioisotopes and a method of preparing the same.

In order to achieve the above object, the present invention provides hydrogel microparticles represented by Chemical formula 1 and having a cross-linked structure of hyaluronic acid polymer compounds having a linker functional group capable of binding to a cationic radioisotope, and having a particle shape having a micro-scale size:

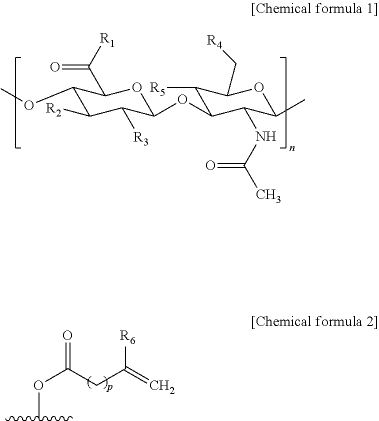

in the Chemical formula 1, n is an integer of 10 to 10,000, R₁ represents the linker functional group, at least one of R₂ to R₅ includes a substituent of chemical formula 2 and rest are hydroxy groups, and
in the Chemical formula 2, p is an integer of 0 to 10, and R₆ is an alkyl group having 1 to 5 carbon atoms.

Also, the present invention provides a method of preparing hydrogel microparticles, comprising preparing a compound represented by the Chemical formula 1 by reacting a hyaluronic acid polymer and a linker capable of binding to a cationic radioisotope; forming microdroplets by flowing a mixed solution of the compound represented by the Chemical formula 1 and an initiator into an oil phase; and freeze-drying the microdroplets.

In addition, the present invention provides microparticles for radiotherapy comprising hydrogel microparticles represented by Chemical formula 1 and having a structure in which hyaluronic acid polymer compounds are cross-linked; and a cationic radioisotope bound to R₁ of the Chemical formula 1.

The hydrogel microparticles according to the present invention have the advantage of being labelable with a cationic radioisotope having high medical utility, and homogeneous hydrogel microparticles can be manufactured through a single process, and there is an advantage in that long-term storage and distribution of hydrogel microparticles is possible through freeze-drying.

### BRIEF DESCRIPTION OF THE DRAWINGS

FIG. 1 shows a schematic diagram illustrating a method of manufacturing hydrogel microparticles according to one embodiment of the present invention.

FIG. 2 shows a flow chart illustrating a method of manufacturing hydrogel microparticles according to one embodiment of the present invention.

FIG. 3 shows a ¹H nuclear magnetic resonance spectroscopy spectrum graph of hyaluronic acid methacrylate (HAMA) included in hydrogel microparticles according to one embodiment of the present invention.

FIG. 4 shows an optical microscope image (a) and a particle distribution measurement graph (b) of hydrogel microparticles according to one embodiment of the present invention.

FIG. 5 shows a scanning electron microscope image of hydrogel microparticles according to one embodiment of the present invention.

FIG. 6 shows a graph showing the labeling efficiency (a) and labeling stability evaluation (b) of cationic metals of hydrogel microparticles according to one embodiment of the present invention.

### DETAILED DESCRIPTION OF THE INVENTION

Hereinafter, examples of the present invention will be described in detail with reference to the attached drawings. The present invention can be modified in various ways and can have various forms, and thus specific examples are illustrated in the drawings and described in detail in the text. However, it is not intended to limit the present invention to a specific disclosed form, but should be understood to include all modifications, equivalents, or substitutes included in the spirit and technical scope of the present invention. In describing each drawing, similar reference numerals are used for similar components. In the attached drawings, the dimensions of structures are illustrated larger than actual dimensions in order to ensure clarity of the present invention.

The terms such as first, second, etc. may be used to describe various components, but the components should not be limited by the terms. The terms are used only for the purpose of distinguishing one component from another. For example, without departing from the scope of the present invention, the first component may be referred to as the second component, and similarly, the second component may also be referred to as the first component.

The terminology used in the present invention is only used to describe specific examples and is not intended to limit the present invention. The singular expression includes the plural expression unless the context clearly indicates otherwise. In this application, it should be understood that the terms “comprise” or “have” and the like are intended to specify the presence of a feature, number, step, operation, component, part, or combination thereof described in the specification, but do not exclude in advance the possibility of the presence or addition of one or more other features, numbers, steps, operations, components, parts, or combinations thereof.

Meanwhile, unless otherwise defined, all terms used herein, including technical or scientific terms, have the same meaning as commonly understood by a person of ordinary skill in the art to which the present invention belongs. Terms defined in commonly used dictionaries, such as those defined in common dictionaries, should be interpreted as having a meaning consistent with the meaning they have in the context of the relevant art, and shall not be interpreted in an idealized or overly formal sense, unless explicitly defined in this application.

The hydrogel microparticles according to the present invention may be represented by the following Chemical formula 1, and have a cross-linked structure of hyaluronic acid polymer compounds having a linker functional group capable of binding to a cationic radioisotope, and may be in the form of particles having a micro-scale size:

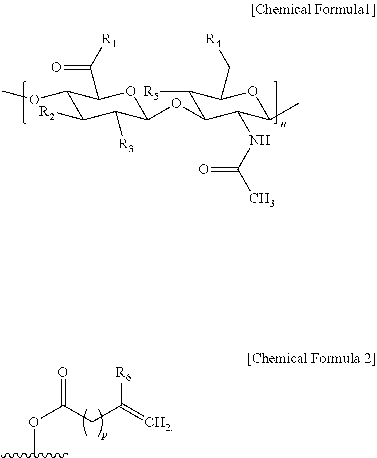

In the Chemical formula 1, n is an integer of 10 to 10,000 or an integer of 50 to 3,000, R₁ represents the linker functional group, at least one of R₂ to R₅ includes a substituent of the Chemical formula 2 and the rest are hydroxy groups.

In the Chemical formula 2, p is an integer of 0 to 10, an integer of 0 to 5 or an integer of 0 to 3, and R₆ is an alkyl group having 1 to 5 carbon atoms or an alkyl group having 1 to 3 carbon atoms.

Specifically, in the hyaluronic acid polymer compound represented by the Chemical formula 1, the substituent of the Chemical formula 2 may be a photocrosslinkable functional group. More specifically, the substituent of the Chemical formula 2 may be at least one of methacrylate, butylacrylate and pentacrylate, for example, methacrylate.

The substitution rate of the substituent of the Chemical formula 2 may be 40 to 80%, 50 to 70%, or 55 to 65%. By having such a substitution rate of the substituent, the crosslinking degree of the crosslinked hydrogel can be controlled to form porous hydrogel microparticles.

The linker functional group may comprise at least one selected from the group consisting of deferoxamine, a DTPA derivative (p-NH₂-Bn-DTPA), a DOTA derivative (NH₂-PEG₄-DOTA, NH₂-GA-DOTA), N-(3-aminopropyl)imidazole, 3-(pyrrol)-1-propanamine, N-(3-aminopropyl)pyridine, and tyramine. As described above, a linker functional group capable of binding to a cationic radioisotope can be included to stably label the cationic radioisotope inside the hydrogel microparticles.

The hydrogel microparticles may have a porous structure.

As described above, a wide surface can be provided to rapidly absorb drugs including radioisotopes into the hydrogel, and the loading amount can be increased by having the porous structure.

The hydrogel microparticle may be a spherical particle having an average diameter of 50 to 100 μm, 65 to 95 μm, or 75 to 90 μm.

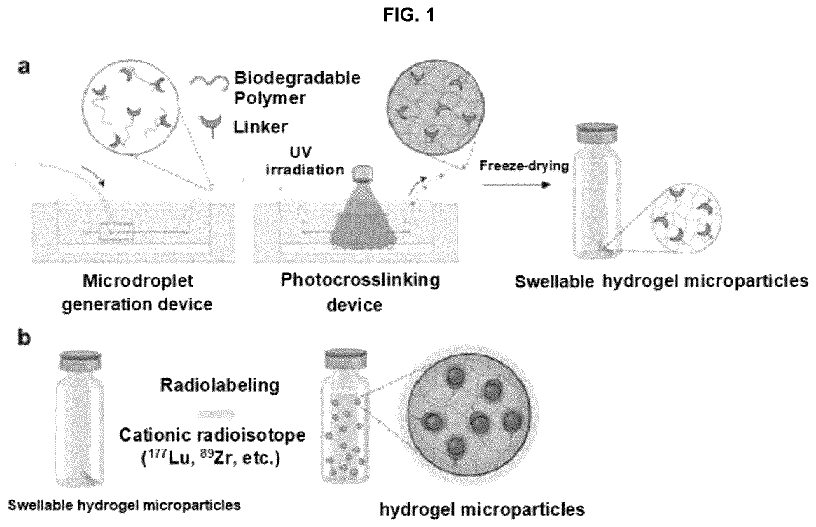

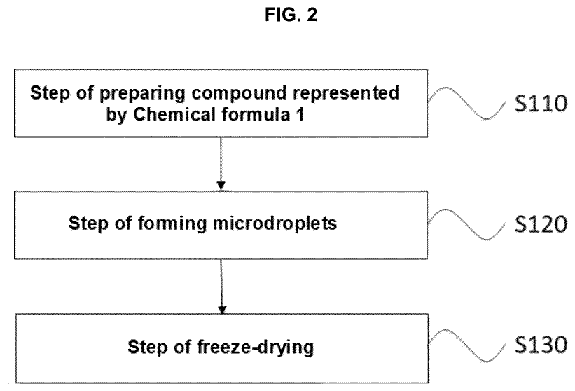

FIG. 1 is a schematic diagram illustrating a method of manufacturing hydrogel microparticles according to one embodiment of the present invention, and FIG. 2 is a flowchart illustrating a method of manufacturing hydrogel microparticles according to one embodiment of the present invention.

Referring to FIG. 1 and FIG. 2, the method of preparing hydrogel microparticles according to the present invention may comprise a step S110 of preparing a compound represented by Chemical formula 1 by reacting a hyaluronic acid polymer and a linker capable of binding to a cationic radioisotope; a step S120 of forming microdroplets by flowing a mixed solution of the compound represented by the Chemical formula 1 and an initiator into an oil phase; and a step S130 of freeze-drying the microdroplets.

The step S110 of preparing the compound represented by the Chemical formula 1 may be performed by mixing a hyaluronic acid polymer, a linker, and a coupling agent to bind the linker to the hyaluronic acid polymer to prepare the compound represented by the Chemical formula 1.

Specifically, the hyaluronic acid polymer may be a compound represented by the following Chemical formula 1-1:

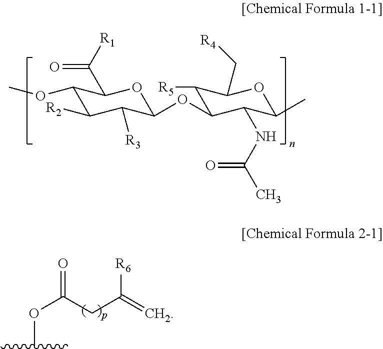

In the Chemical formula 1-1, n is an integer of 50 to 3,000, R₁ is a hydroxy group, at least one of R₂ to R₅ includes a substituent of Chemical formula 2 and the rest are hydroxy groups.

In the Chemical formula 2-1, p is an integer of 0 to 10, and R₆ is an alkyl group having 1 to 5 carbon atoms.

The substituent of the Chemical formula 2-1 may be a photocrosslinkable functional group. More specifically, the substituent of the Chemical formula 2-1 may be at least one of methacrylate, butylacrylate, and pentacrylate, for example, methacrylate.

The substitution rate of the substituent of the Chemical formula 2-1 may be 40 to 80%, 50 to 70%, or 55 to 65%. By having such a substitution rate of the substituent, the crosslinking degree of the crosslinked hydrogel can be controlled, thereby forming porous hydrogel microparticles.

The linker may comprise at least one selected from the group consisting of deferoxamine, a DTPA derivative (p-NH₂-Bn-DTPA), a DOTA derivative (NH₂-PEG₄-DOTA, NH₂-GA-DOTA), N-(3-aminopropyl)imidazole, 3-(pyrrol)-1-propanamine, N-(3-aminopropyl)pyridine, and tyramine.

The coupling agent may be 1-ethyl-3-(3-dimethylaminopropyl)carbodiimide (EDC) and N-hydroxysuccinimide (NHS).

The step S120 of forming the microdroplets can form polymer microdroplets dispersed in the oil by flowing the mixed solution together with the oil phase into the microdroplet generation device.

Specifically, in the step S120 of forming the microdroplets, the mixed solution and the oil phase can be injected into the microdroplet generation device at a volume ratio of 1:2 to 6 or 1:3 to 5. In a section where a rapid change in the channel cross-section occurs as the polymer solution passes through a thin microchannel, polymer microdroplets can be spontaneously formed by changes in interfacial tension and flow characteristics. The droplets formed in this process can be dispersed in the oil phase to obtain polymer microdroplets.

Specifically, the step S120 of forming the microdroplets may irradiate UV light to the polymer microdroplets manufactured along the microfluidic device to crosslink the hydrogel microparticles. The UV light irradiation conditions may be irradiated with UV light of 250 to 500 mW/cm² or 300 to 400 mW/cm² for 30 seconds to 5 minutes or 1 minute to 3 minutes to perform the crosslinking reaction.

The photoinitiator may comprise at least one of lithium phenyl-2,4,6-trimethylbenzoylphosphinate, lithium benzoyl(phenyl)phosphinate, and magnesium benzoyl(phenyl)phosphinate.

The oil phase may comprise at least one selected from the group consisting of NOVEC® 7500, Fluo-Oil 135, Fluorinert® FC-40, perfluoropolyether (PFPE)-based oil, silicone oil, and mineral oil.

The oil phase may further include a surfactant, and the surfactant may comprise at least one selected from the group consisting of Pico-Surf, FluoSurf, Krytox®-based surfactants, and fluorinated surfactants.

The freeze-drying step S130 may prepare porous hydrogel microparticles by freeze-drying the microdroplets at −50° C. to 0° C. or −30° C. to −10° C. for 30 to 60 hours or 45 to 55 hours.

After the freeze-drying step, a step of mixing the freeze-dried microparticles with a cationic radioisotope to prepare microparticles for radiotherapy may be additionally included.

The cationic radioisotopes may comprise at least one selected from the group consisting of ruthenium-177, zirconium-89, gallium-68, yttrium-90, indium-111, copper-64, gadolinium-153, technetium-99m, and actinium-225.

The microparticles for radiotherapy according to the present invention may comprise a hydrogel microparticle having a structure in which hyaluronic acid polymer compounds are cross-linked, represented by the Chemical formula 1; and a cationic radioisotope bound to R₁ of the Chemical formula 1.

The cationic radioisotope may comprise at least one selected from the group consisting of ruthenium-177, zirconium-89, gallium-68, yttrium-90, indium-111, copper-64, gadolinium-153, technetium-99m, and actinium-225.

The linker functional group may comprise at least one selected from the group consisting of deferoxamine, a DTPA derivative (p-NH₂-Bn-DTPA), a DOTA derivative (NH₂-PEG₄-DOTA, NH₂-GA-DOTA), N-(3-aminopropyl)imidazole, 3-(pyrrol)-1-propanamine, N-(3-aminopropyl)pyridine, and tyramine.

Hereinafter, the present invention will be described in more detail through examples. These examples are intended to explain the present invention in more detail, and the scope of the present invention is not limited to these examples. The examples of the present invention are provided to more completely explain the present invention to a person having average knowledge in the art.

### Example 1

### Manufacturing Example 1

Hyaluronic acid methacrylate (HAMA) with different degrees of crosslinking was synthesized using hyaluronic acid (HA), a biodegradable polymer. First, 5 g of HA was added to 100 mL of deionized water and stirred at 400 RPM (rotation per minute) using a centrifugal mixer for 3 hours at 25° C. to prepare an HA solution. After cooling the solution to 4° C., the pH was adjusted to 8 using 1 M sodium hydroxide (NaOH) solution. Methacrylic anhydride was added in 1 and 4 equivalents (2.1 and 8.4 g) per hyaluronic acid (HA) disaccharide unit, and the reaction was carried out so that the degree of methacrylation (DM) of methacrylate (MA) was different. To maintain the pH at 8-10 during the reaction, NaOH solution was additionally used, and the reaction was performed at 4° C. for 24 hours. After the reaction, to remove unreacted substances, dialysis was performed for 96 hours using a cellulose membrane with a MWCO (molecular weight cut-off: 14,000), and deionized water was replaced every 8 hours. Finally, HAMA powder was obtained by freeze-drying.

### Manufacturing Example 2

For efficient labeling of drugs or radioisotopes, as shown in the following reaction formula, an experiment was conducted to bind deferoxamine (DFO) to HAMA with degree of methacrylation of 60% or 120% prepared in Manufacturing Example 1.

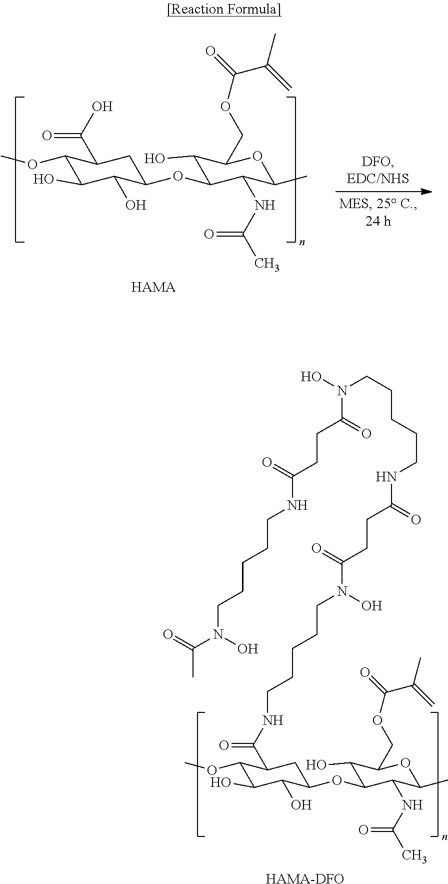

To couple DFO acting as a linker, to HAMA, a coupling reaction using 1-ethyl-3-(3-dimethylaminopropyl)carbodiimide (EDC) and N-hydroxysuccinimide (NHS) was utilized. First, 500 mg (0.9 mmol) of each HAMA was dissolved in 20 mL of 2-(N-morpholino)ethanesulfonic acid (MES) buffer (pH 6.0). To this solution, 86 mg (0.45 mmol) of EDC and 103 mg (0.9 mmol) of NHS were added, and the solution was stirred at room temperature for 30 min to activate the carboxyl group of HAMA. Thereafter, 252 mg (0.45 mmol) of DFO, a linker for labeling radioisotopes, was added to the reaction solution, and stirred at room temperature for 24 hours to induce the binding of HAMA and DFO. After completion of the reaction, the mixture was dialyzed against distilled water using a cellulose membrane (MWCO: 14,000) for 72 hours to remove unreacted DFO and residual reagents, and the water was replaced every 8 hours. Finally, HAMA with DM 60% and 120% bound to the linker (DFO) was obtained through freeze-drying, which was intended to be utilized for efficient labeling of drugs or radioisotopes.

### Example 1

Swellable microparticles with different degrees of crosslinking were prepared using HAMA with controlled methacrylation rate. HAMA was dissolved in deionized water at a concentration of 5% (w/v), and 0.5% (w/v) of lithium phenyl-2,4,6-trimethylbenzoylphosphinate (LAP), which is a photoinitiator, was added and mixed. The oil phase was prepared by mixing 50 mL of Novec7500 with 1 mL (2% v/v) of surfactant Pico-surf. HAMA solution of 1 mL was flowed into the microdroplet production device at a volume ratio of 1:4 into the oil phase to form HAMA microdroplets as an oil-in-water (W/O) emulsion. HAMA microdroplets were irradiated with UV light of 405 nm wavelength at 350 mW/cm² for 1 minute to induce a crosslinking reaction, and a HAMA hydrogel microparticle solution was obtained through a purification process using ethanol and deionized water. Finally, the HAMA hydrogel microparticle solution was frozen at −20° C. and then freeze-dried at −50° C. for 48 hours to obtain absorbent hydrogel microparticles according to the degree of crosslinking.

### Example 2

HAMA (DM 60, 120%) coupled with the linker (DFO) manufactured in Manufacturing Example 2 was dissolved in deionized water at a concentration of 5% (w/v), and 0.5% (w/v) of lithium phenyl-2,4,6-trimethylbenzoylphosphinate (LAP), which is a photoinitiator, was added and mixed. The oil phase was prepared by mixing 100 mL of Novec7500 with 0.5 mL (0.5% v/v) of surfactant Pico-surf. The HAMA-DFO solution of 1 mL of was flowed into the microdroplet production device at a volume ratio of 1:4 into the oil phase to form HAMA-DFO microdroplets as an oil-in-water (W/O) emulsion. HAMA-DFO microdroplets were irradiated with UV light of 405 nm wavelength at 350 mW/cm² for 1 minute to perform a crosslinking reaction, and a HAMA-DFO hydrogel microparticle solution was obtained through a purification process using ethanol and deionized water. Finally, the HAMA-DFO hydrogel microparticle solution was frozen at −20° C. and then freeze-dried at −50° C. for 48 hours to obtain absorbent microparticles according to the degree of crosslinking.

### Experimental Example 1

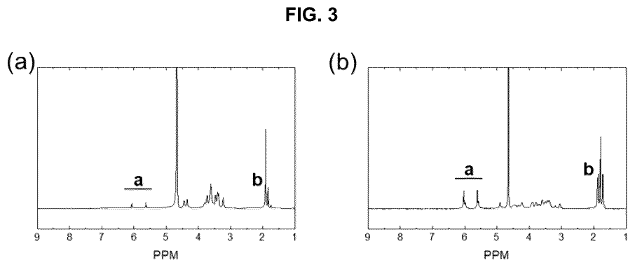

In order to confirm the degree of methacrylation (DM) of the synthesized hyaluronic acid methacrylate (HAMA), ¹H nuclear magnetic resonance spectroscopy (NMR) analysis of the hyaluronic acid methacrylate manufactured in Manufacturing Example 1 was performed, and the results are shown in FIG. 3.

FIG. 3A shows the H nuclear magnetic resonance spectrum of hyaluronic acid methacrylate (HAMA) with degree of methacrylation (DM) of 60%, and FIG. 3B shows the H nuclear magnetic resonance spectrum of hyaluronic acid methacrylate (HAMA) with a degree of methacrylation (DM) of 120%.

As shown in FIG. 3, the acrylate proton (C═CH₂) of the methacrylate group was observed at δ=5.6 and 6.1 ppm in the methacrylic spectrum, and the methyl proton (C(═O)CH₃) of hyaluronic acid appeared at δ=1.9 ppm. The degree of methacrylation was calculated using the integral value ratio of the acrylate proton to the methyl proton of HA. Through this, the DM of HAMA was confirmed to be 60 and 120% according to the amount of methacrylic anhydride added.

### Experimental Example 2

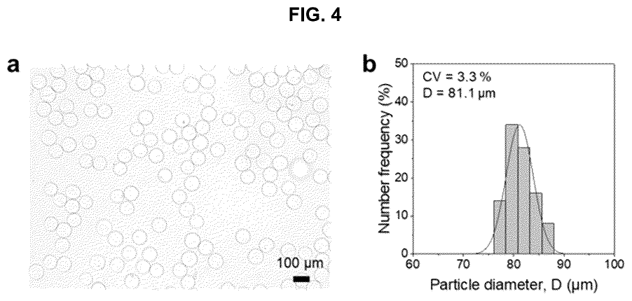

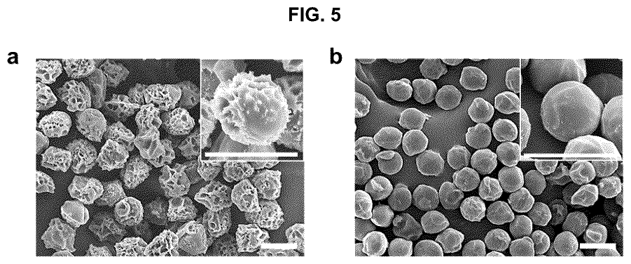

In order to confirm the shape of the hydrogel microparticles manufactured in Example 1, the particle distribution of the fine particles was measured by photographing with an optical microscope and a scanning electron microscope, and the results are shown in FIG. 4 and FIG. 5.

FIG. 4 is an optical microscope photograph (A) and a particle distribution measurement graph (B) of the hydrogel microparticles manufactured in Example 1. As shown in FIG. 4A, which is the optical microscope photograph of the hydrogel microparticles manufactured using the photocrosslinking microfluidic technique, it was confirmed that they had a uniform shape and size. In addition, looking at FIG. 4B, the diameter of the microparticles was measured and calculated using the coefficient of variation (CV), which is the standard deviation divided by the mean. As a result, the diameter of the microparticles was 75 to 90 μm, and it was confirmed that monodisperse hydrogel microparticles were formed at 3.3%.

Looking at FIG. 5, it was confirmed that the hydrogel microparticles (b) with a high degree of methacrylation (DM) maintained a smooth spherical shape even after freeze-drying, while the hydrogel microparticles with a low degree of methacrylation (DM) had a porous structure with a large surface area after freeze-drying. Therefore, drugs including radioisotopes can be rapidly absorbed into the hydrogel microparticles, thereby increasing the loading amount. The freeze-dried hydrogel microparticles were photographed using a scanning electron microscope (SEM) (FIG. 5A), and the specific surface area was calculated. Compared to the microparticles without a porous structure on the surface (FIG. 5B), they had a large surface area of 50 to 100% or more.

### Experimental Example 3

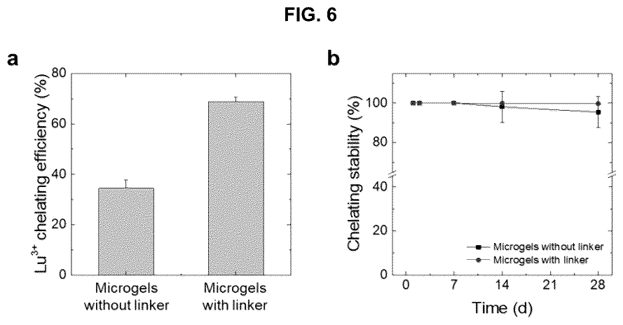

In order to confirm the cationic metal labeling efficiency of the hydrogel microparticles, the cationic metal labeling efficiency was analyzed using the hydrogel microparticles manufactured in Example 2, and the results are shown in FIG. 6.

As shown in FIG. 6A, the labeling efficiency evaluation results show that the group with the linker (DFO) showed a higher labeling efficiency of 68.8±1.9% compared to the group without the linker (DFO) (34.5±3.3%). This suggests that it has the potential for radiolabeling various cationic radioisotopes used in the medical field (e.g., lutetium-177, zirconium-89, gallium-68, etc.).

Looking at FIG. 6B, an experiment was conducted to analyze the performance of stably labeling cationic metals (lutetium-177, zirconium-89, gallium-68) inside hydrogel microparticles, and it was found that hydrogel microparticles containing linkers were shown to stably label cationic metals internally over time.

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

1. Hydrogel microparticles represented by Chemical formula 1 and having a crosslinked structure of hyaluronic acid polymer compounds having a linker functional group capable of binding to a cationic radioisotope, and having a particle shape with a micro-scale size: 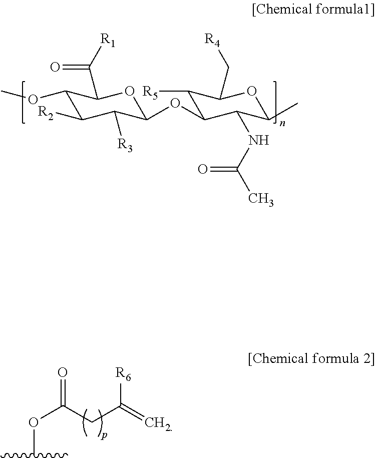
in the Chemical formula 1, n is an integer of 10 to 10,000, R₁ represents the linker functional group, at least one of R₂ to R₅ includes a substituent of chemical formula 2 and rest are hydroxy groups, and
in the Chemical formula 2, p is an integer of 0 to 10, and R₆ is an alkyl group having 1 to 5 carbon atoms.

2. The hydrogel microparticles of claim 1, wherein substitution rate of the substituent of the Chemical formula 2 is 40 to 80%.

3. The hydrogel microparticles of claim 1, wherein the hydrogel microparticles have a porous structure.

4. The hydrogel microparticles of claim 3, wherein the hydrogel microparticles have a spherical particle with a diameter of 50 to 100 m.

5. The hydrogel microparticles of claim 1, wherein the linker functional group comprises at least one selected from the group consisting of deferoxamine, a DTPA derivative (p-NH₂-Bn-DTPA), a DOTA derivative (NH₂-PEG₄-DOTA, NH₂-GA-DOTA), N-(3-aminopropyl)imidazole, 3-(pyrrol)-1-propanamine, N-(3-aminopropyl)pyridine, and tyramine.

6. A method of preparing hydrogel microparticles, comprising
preparing a compound represented by Chemical formula 1 by reacting a hyaluronic acid polymer and a linker capable of binding to a cationic radioisotope;
forming microdroplets by flowing a mixed solution of the compound represented by the Chemical formula 1 and an initiator into an oil phase; and
freeze-drying the microdroplets: 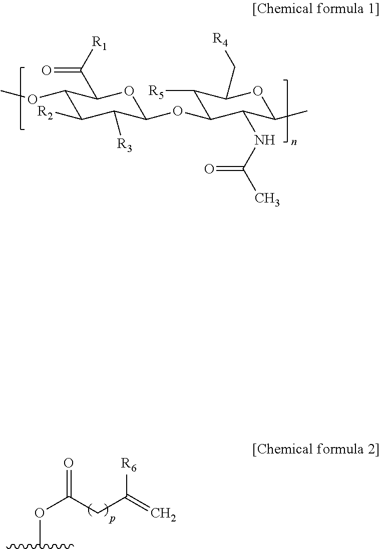
in the Chemical formula 1, n is an integer of 10 to 10,000, R₁ represents the linker functional group, at least one of R₂ to R₅ includes a substituent of Chemical formula 2 and rest are hydroxy groups, and
in the Chemical formula 2, p is an integer of 0 to 10, and R₆ is an alkyl group having 1 to 5 carbon atoms.

7. The method of preparing hydrogel microparticles of claim 6, wherein substitution rate of the substituent of the Chemical formula 2 is 40 to 80%.

8. The method of preparing hydrogel microparticles of claim 6, wherein forming the microdroplets is characterized in that the mixed solution is flowed together with an oil phase into a microdroplet production device to form polymer microdroplets dispersed in oil.

9. The method of preparing hydrogel microparticles of claim 6, wherein the freeze-drying is characterized in that the microdroplets are freeze-dried at −50° C. to 0° C. for 30 to 60 hours.

10. The method of preparing hydrogel microparticles of claim 6, further comprising producing microparticles for radiotherapy by mixing freeze-dried hydrogel microparticles with a cationic radioisotope after the freeze-drying.

11. Microparticles for radiotherapy comprising:
hydrogel microparticles represented by Chemical formula 1 and having a structure in which hyaluronic acid polymer compounds are cross-linked; and
a cationic radioisotope bound to R₁ of the Chemical formula 1: 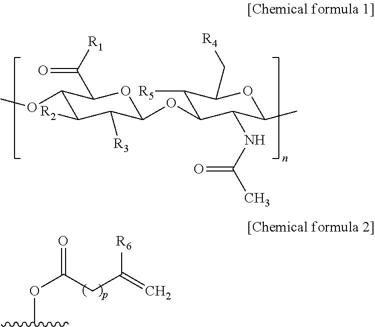
in the Chemical formula 1, n is an integer of 10 to 10,000, R₁ represents the linker functional group, at least one of R₂ to R₅ includes a substituent of Chemical formula 2 and rest are hydroxy groups, and
in the Chemical formula 2, p is an integer of 0 to 10, and R₆ is an alkyl group having 1 to 5 carbon atoms.

12. The microparticles for radiotherapy of claim 11, wherein the cationic radioisotope is at least one selected from the group consisting of ruthenium-177, zirconium-89, gallium-68, yttrium-90, indium-111, copper-64, gadolinium-153, technetium-99m, and actinium-225.

13. The microparticles for radiotherapy of claim 11, wherein the linker functional group comprises at least one selected from the group consisting of deferoxamine, a DTPA derivative (p-NH₂-Bn-DTPA), a DOTA derivative (NH₂-PEG₄-DOTA, NH₂-GA-DOTA), N-(3-aminopropyl)imidazole, 3-(pyrrol)-1-propanamine, N-(3-aminopropyl)pyridine, and tyramine.
